(19) United States
(12) Patent Application Publication
(10) Pub. No.: US 2026/0061184 A1
(43) Pub. Date: Mar. 5, 2026

(54) PERCUTANEOUS BLOOD PUMP WITH ARTICULATING RIGID LENGTH

(71) Applicant: BOSTON SCIENTIFIC SCIMED, INC., Maple Grove, MN (US)

(72) Inventors: Daniel Presteng, Corcoran, MN (US); Darrin Dale Beekman, Osseo, MN (US); Arden Michael Thomas Costello, Maple Grove, MN (US)

(73) Assignee: BOSTON SCIENTIFIC SCIMED, INC., Maple Grove, MN (US)

(21) Appl. No.: 19/310,274

(22) Filed: Aug. 26, 2025

Related U.S. Application Data
(60) Provisional application No. 63/687,922, filed on Aug. 28, 2024.

(51) Int. Cl. A61M 60/81; A61M 60/165; A61M 60/216; A61M 60/416; A61M 60/90

(52) U.S. Cl. CPC A61M 60/81; A61M 60/165; A61M 60/216; A61M 60/416; A61M 60/90; A61M 2205/0216; A61M 2205/0266; A61M 2205/103; A61M 2210/125

(57) ABSTRACT

A percutaneous circulatory support system may include a blood pump. The blood pump may include an impeller assembly with an impeller housing having a first central axis. The blood pump may include a motor housing having a second central axis. A magnetic assembly may magnetically couple the impeller assembly with the motor assembly. A flexible connector may couple the impeller housing with the motor housing. The magnetic assembly may bias the impeller housing and the motor housing to a first configuration with the first central axis aligned with the second central axis.

### CROSS REFERENCE TO RELATED APPLICATIONS

The present application claims the benefit of U.S. Provisional Patent Application Ser. No. 63/687,922, filed Aug. 28, 2024, the disclosure of which is incorporated herein by reference.

### TECHNICAL FIELD

The present disclosure pertains to a circulatory support device or system. More particularly, the present disclosure pertains to a percutaneous circulatory support device or system including an articulating connection between rigid portions of the circulatory support device or system.

### BACKGROUND

Percutaneous mechanical circulatory support devices, such as blood pumps can provide transient support for hours or months of use in patients whose heart function or cardiac output is compromised. The percutaneous mechanical circulatory support devices may be navigated through the vasculature to a patient's heart. Such devices may be navigated through the aortic arch and placed across the aortic valve, for example. Various configurations of percutaneous mechanical circulatory support devices are known. However, there is an ongoing need to provide improved construction of percutaneous mechanical circulatory support devices.

### BRIEF SUMMARY

This disclosure provides design, material, manufacturing method, and use alternatives for medical devices, including percutaneous circulatory support devices or systems and associated percutaneous blood pumps.

A first example may include a blood pump having an impeller assembly comprising an impeller housing having a first central axis, a motor assembly comprising a motor housing having a second central axis, a magnetic assembly magnetically coupling the impeller assembly with the motor assembly, and a flexible connector coupling the impeller housing with the motor housing. The magnetic assembly may be configured to bias the impeller housing and the motor housing to a first configuration with the first central axis aligned with the second central axis.

Alternatively or additionally to any of the examples above, in another example, the impeller housing may be rigid and the motor housing may be rigid.

Alternatively or additionally to any of the examples above, in another example, the flexible connector may be formed from an elastomer material configured to bias the impeller housing and the motor housing to the first configuration.

Alternatively or additionally to any of the examples above, in another example, the flexible connector may be an elastomer tube.

Alternatively or additionally to any of the examples above, in another example, the flexible connector may be formed from a nickel-titanium alloy configured to bias the impeller housing and the motor housing to the first configuration.

Alternatively or additionally to any of the examples above, in another example, the flexible connector may be configured to facilitate the impeller housing and the motor housing adjusting to a second configuration in which the first central axis is transverse to the second central axis.

Alternatively or additionally to any of the examples above, in another example, the flexible connector may be configured to limit an angle between the first central axis and the second central axis when the impeller housing and the motor housing are in the second configuration.

Alternatively or additionally to any of the examples above, in another example, a distal end of the motor housing may have a first radius of curvature and a proximal end of the impeller housing may have a second radius of curvature configured to mate with the first radius of curvature.

Alternatively or additionally to any of the examples above, in another example, the blood pump may further include a tube, wherein the tube may be configured to adjust from a first position in which the tube is distal of the flexible connector and a second position in which the tube extends along the impeller housing and the motor housing to maintain the first central axis in alignment with the second central axis.

Alternatively or additionally to any of the examples above, in another example, the impeller assembly may comprise an impeller within the impeller housing and the motor assembly comprises a motor within the motor housing.

Alternatively or additionally to any of the examples above, in another example, the magnetic assembly may comprise a first magnet within the impeller housing and coupled with an impeller shaft coupled with the impeller and a second magnet within the motor housing and coupled with a drive shaft coupled with the motor.

Alternatively or additionally to any of the examples above, in another example, a flexible elongate shaft may extend in a proximal direction from the motor assembly.

In a further example, a blood pump may include an impeller assembly comprising a first magnet, an impeller shaft coupled with the first magnet, an impeller coupled with the impeller shaft, and an impeller housing that houses the first magnet, the impeller shaft, and the impeller, a motor assembly comprising a second magnet, a drive shaft coupled with the second magnet, a motor coupled with the drive shaft, and a motor housing that houses the second magnet, the drive shaft, and the motor, and a resilient connector coupling the impeller housing with the motor housing and configured to bias the impeller housing and the motor housing to an axial configuration and allow the impeller housing and the motor housing to articulate relative to one another.

Alternatively or additionally to any of the examples above, in another example, the first magnet and the second magnet may be configured to bias the impeller housing and the motor housing to the axial configuration.

Alternatively or additionally to any of the examples above, in another example, the resilient connector may have a length configured to maintain an operative distance between the first magnet and the second magnet while the resilient connector adjusts between a first configuration and a second configuration.

Alternatively or additionally to any of the examples above, in another example, the resilient connector may be formed from an elastomer material.

Alternatively or additionally to any of the examples above, in another example, a distal end of the motor housing may have a first radius of curvature and a proximal end of the impeller housing may have a second radius of curvature configured to mate with the first radius of curvature.

In a further example, a method of using a blood pump may include inserting the blood pump into vasculature of a patient, the blood pump comprising a motor housing and an impeller housing configured to articulate relative to one another, advancing the blood pump through the vasculature, wherein the motor housing and the impeller housing are configured to articulate relative to one another around curvatures in the vasculature, and positioning the blood pump at a location proximate an aortic valve of the patient, wherein the motor housing and the impeller housing are configured to axially align with one another at the location proximate the aortic valve.

Alternatively or additionally to any of the examples above, in another example, the motor housing and the impeller housing may be coupled to one another via a magnetic coupling and the magnetic coupling may bias the motor housing and the impeller housing to an axially aligned configuration.

Alternatively or additionally to any of the examples above, in another example, the blood pump may include a flexible connector coupled with the motor housing and the impeller housing, the flexible connector may be configured to limit articulation between the motor housing and the impeller housing.

The above summary of some configurations is not intended to describe each disclosed configuration or every implementation of the present disclosure. The Figures, and Detailed Description, which follow, more particularly illustrate some of these configurations.

### BRIEF DESCRIPTION OF THE DRAWINGS

The disclosure may be more completely understood in consideration of the following detailed description in connection with the accompanying drawings, in which:

FIG. 1 is a schematic perspective view of an illustrative mechanical circulatory support (MCS) system;

FIG. 2 is a schematic partial cross-section view of anatomy and a schematic side view of an illustrative MCS system within the anatomy;

FIG. 3 is a perspective view of a distal portion of an illustrative MCS system;

FIG. 4 is a schematic cross-section view of a portion of an introducer sheath within a blood vessel and a portion of an illustrative MCS system in the introducer sheath and the blood vessel;

FIG. 5 a schematic side view of a portion of an illustrative percutaneous blood pump of an MCS system;

FIGS. 6A and 6B are schematic cross-section views of the portion of the illustrative percutaneous blood pump depicted in FIG. 5;

FIGS. 7A and 7B are schematic cross-section views of a portion of an illustrative percutaneous blood pump of an MCS system;

FIGS. 8A-8C are schematic cross-section views of a portion of an illustrative percutaneous blood pump of an MCS system; and

FIG. 9 is a schematic diagram of an illustrative technique for using a percutaneous blood pump of an MCS system.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

A variety of circulatory assist or support devices are known for assisting or replacing a pumping function of a heart in a patient with severe heart failure and/or other cardiac conditions. Circulatory support devices may be configured to treat patients with cardiogenic shock, myocardial infarction, acutely decompensated heart failure, and/or other heart related conditions. Additionally or alternatively circulatory support systems or devices may support a patient during percutaneous coronary interventions and/or other procedures.

Example cardiac circulatory support systems or devices include, but are not limited to, ventricular assist devices (VADs), total artificial hearts, intra-aortic balloon pumps (IABP), and extracorporeal membrane oxygenation (ECMO) devices. Example VADs include left ventricular assist devices (LVADs), right ventricular assist devices (RVADs), and biventricular assist devices (BiVADs). A further illustrative VAD is a percutaneous ventricular assist device (PVAD), which may be inserted into a ventricle (e.g., a left ventricle or a right ventricle) of a heart of a patient via delivery through an access site to a femoral artery or vein and/or other suitable vasculature in communication with the ventricle. A PVAD may be placed at a desired location of anatomy of a patient via percutaneous access and delivery, which may enable the PVAD to be used in emergency medicine, a cath lab, and/or other surgical and/or non-surgical settings.

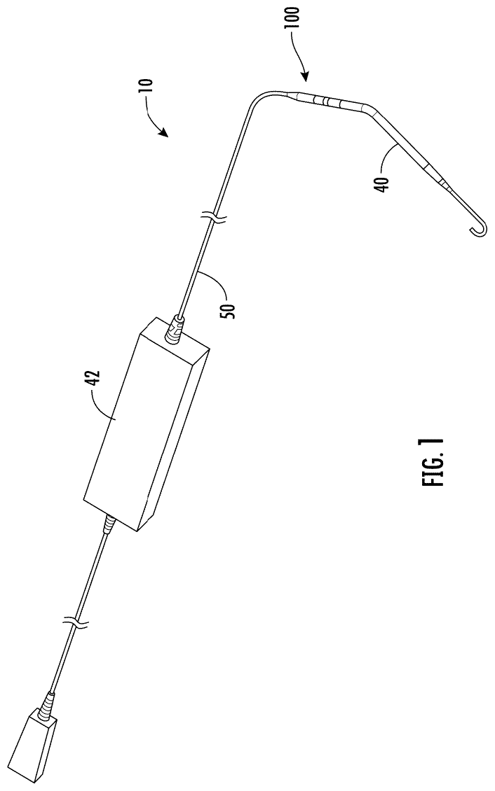

FIG. 1 depicts a schematic view of an illustrative circulatory support system 10 (e.g., a mechanical circulatory support (MCS) system). The system 10 may include a percutaneous support device (e.g., a PVAD, such as a blood pump 100), a cannula 40, an elongate shaft or tube 50, and an introducer sheath (not shown). In some examples, the system 10 may include a guidewire, but this is not required. The introducer sheath, when included, may facilitate percutaneous delivery of the blood pump 100 and the cannula 40 to a target location within a patient. When positioned at a target site in a heart of a patient, the blood pump 100 may be configured to pump blood from a ventricle of the heart to vasculature of the patient.

The cannula 40 and/or the elongate shaft or tube 50 may be components of the blood pump 100 and/or separate components coupled with the blood pump 100. In some examples, although the cannula 40 and/or the elongate shaft or tube 50 may be discussed as extending from the blood pump 100, the cannula 40 and/or the elongate shaft or tube 50 may be part of the blood pump 100. For example, the cannula 40 and/or the elongate shaft or tube 50 may be formed from a component of the blood pump 100, such as an impeller housing and/or other suitable component. Other suitable configurations are contemplated.

The system 10 may further include a proximal housing 42 coupled with (e.g., connected to) the elongate tube 50, where the elongate tube 50 may be coupled with the blood pump 100 (e.g., a distal end of the elongate tube 50 may be coupled with a proximal end of the blood pump 100, etc.) and one or more wires and/or shafts of, or coupled with, the blood pump 100 may extend proximally from the blood pump 100 through the tube 50. The elongate tube 50 may take the form of a catheter and/or other suitable elongate structure. In some examples, at least the proximal housing 42, the elongate tube 50, and/or other components of the system 10 may be configured to facilitate delivering the blood pump 100 and the cannula 40 to the target location or site within the patient. The elongate tube 50 and the proximal housing 42 may be configured to be positioned at least partially exterior of the patient when the blood pump 100 and the cannula 40 are at the target location. In some examples, the guidewire, when included, may also be used to facilitate the delivery of the blood pump 100 and/or the cannula 40.

Although not shown, the system 10 may additionally or alternatively include one or both of a starter tube and a starter tube flushing line. The starter tube, when included, may couple with a cannula delivery tool (e.g., a delivery sheath) which may be configured to receive the cannula 40 prior to and/or during delivery of the blood pump 100 and the cannula 40 to a target site.

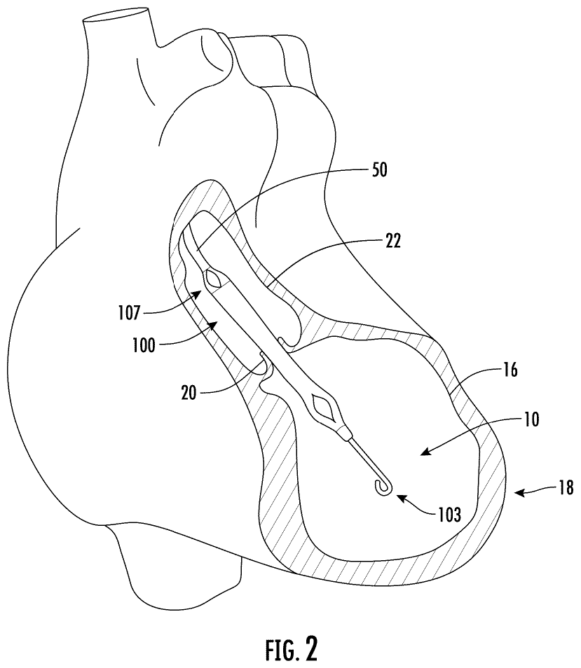

FIG. 2 depicts a schematic view of an illustrative positioning of the blood pump 100 in anatomy of a patient. As depicted in FIG. 2, the blood pump 100 may be positioned with a distal end 103 thereof located in a left ventricle 16 of a heart 18 and a proximal end 107 of the blood pump 100 in an aorta 22, such that the blood pump 100 (e.g., which may or may not include the cannula 40) extends across an aortic valve 20 between the left ventricle 16 and the aorta 22. With the blood pump 100 extending from the left ventricle 16 to the aorta 22 of the patient, the blood pump 100 may be configured to pump blood from the left ventricle 16 into the aorta 22 (e.g., into the ascending aorta) to assist or support blood flow circulation. Other suitable positions of the blood pump 100 relative to the anatomy of the patient are contemplated and include, but are not limited to, the distal end 103 of the blood pump 100 being positioned in or in fluid communication with a right ventricle of the heart 18 with the proximal end 107 of the blood pump 100 being positioned in a pulmonary artery.

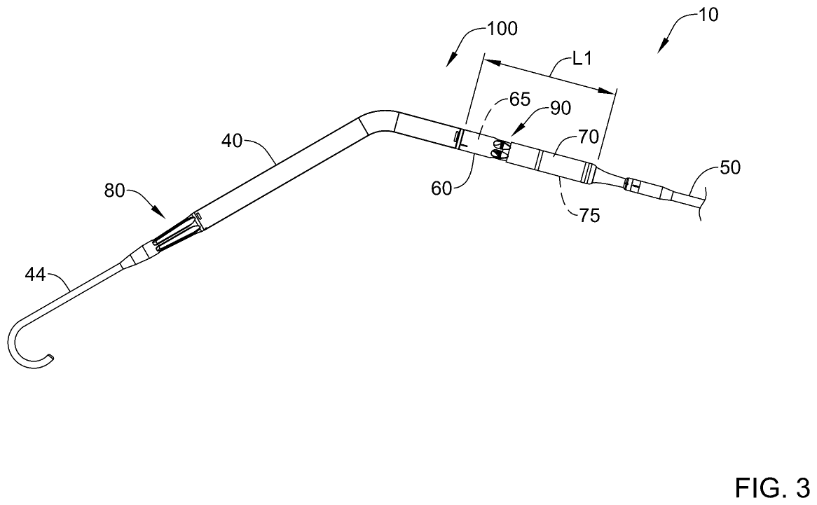

Additional features of the blood pump 100 are illustrated in FIG. 3. The blood pump 100 may generally include the cannula 40 with a distal tip 44 (e.g., a flexible or atraumatic distal tip and/or other suitable distal tip) extending from a distal end of the cannula 40, an impeller housing 60, and a motor housing 70, where the impeller housing 60 and the motor housing 70 have a combined length L1 (e.g., a combined rigid length). The cannula 40 may be flexible or rigid. In some configurations, the cannula 40, the impeller housing 60, and/or the motor housing 70 may be integrally or monolithically constructed. In other instances, the cannula 40, the impeller housing 60, and/or the motor housing 70 may be separate components coupled with or in communication with one another.

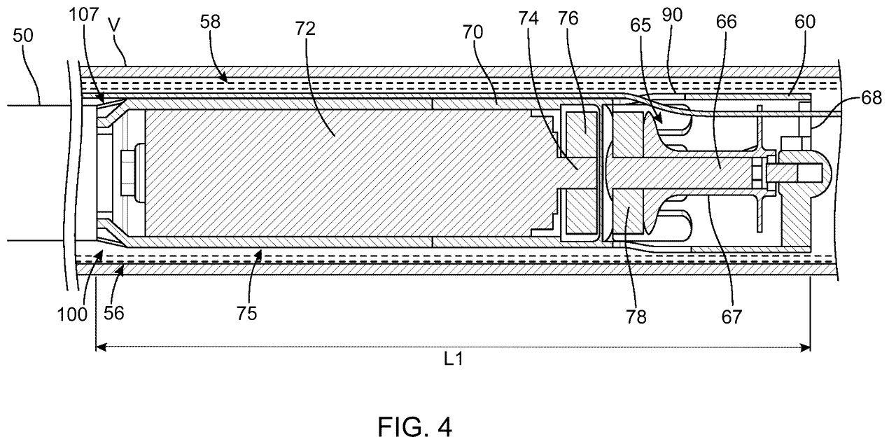

The impeller housing 60 may carry an impeller assembly 65 therein and/or may be part of the impeller assembly 65. The impeller assembly 65 may include an impeller coupled with (e.g., secured to) an impeller shaft, that rotates relative to the impeller housing 60 to drive blood through the blood pump 100, where the impeller and impeller shaft may be within the impeller housing 60. In some configurations, the impeller shaft and the impeller of the impeller assembly 65 may be integrally formed, whereas, in other configurations the impeller shaft and the impeller may be separate components. The impeller and impeller shaft are shown in FIG. 4 and described in greater detail below.

Rotation of the impeller may cause blood to flow from a blood inlet 80 of the blood pump 100, such as at a distal end of the cannula 40, through the cannula 40 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 100 (e.g., at the cannula 40). In some instances, the blood outlet 90 may include a plurality of blood outflow windows arranged around a circumference of the impeller housing 60. In additional and/or alternative configurations, the blood inlet 80 and/or the blood outlet 90 may be formed on other portions of the blood pump 100.

With continued reference to FIG. 3, the motor housing 70 may carry a motor assembly 75 therein and/or may be part of the motor assembly 75. The motor assembly 75 may include a motor configured to rotatably drive the impeller of the impeller assembly 65 relative to the impeller housing 60, wherein the motor may be within the motor housing 70. Electrical power may be supplied to the motor through wiring extending through the elongate tube 50, for example. In some instances, the motor may be physically connected to the impeller. For example, in some configurations the impeller may be mounted on a drive shaft or drive line of the motor. In other configurations, the impeller shaft may be directly or indirectly coupled to the drive shaft of the motor. In some instances, the drive assembly may include a magnetic coupling (e.g., a magnetic assembly) between the motor and the impeller. For example, a drive magnet may be mounted on the drive shaft or drive line of the motor. Rotation of the drive magnet causes rotation of a driven magnet, which is connected to the impeller assembly 65. More specifically, in configurations incorporating an impeller shaft, the impeller shaft and the impeller of the impeller assembly 65 may be configured to rotate with the driven magnet. In other configurations, the motor may be coupled to the impeller assembly 65 via other components.

FIG. 4 depicts a schematic cross-section view of a body portion 58 of an introducer sheath 56 extending into a blood vessel V with the blood pump 100 inserted into the introducer sheath 56. The blood pump 100 may include and/or may be coupled with the elongate tube 50 extending proximally from the proximal end 107 of the blood pump 100 to a location outside of the blood vessel V and the introducer sheath 56. When positioning the blood pump 100 within the patient, the blood pump 100 may be advanced through the blood vessel V and positioned in or at a target location, such as a target cardiac location (e.g., in the aorta 22, across the aortic valve 20, and/or into the left ventricle 16), via the introducer sheath 56. While the introducer sheath 56 is illustrated in FIG. 4 with the use of the blood pump 100, various other medical devices may be used in conjunction with the introducer sheath 56.

The blood pump 100 may include one or more components. In some examples, the blood pump 100 may include an impeller housing 60 and a motor housing 70, which may have the combined length L1. The impeller housing 60 and the motor housing 70 may be integrally or monolithically constructed. Alternatively, the impeller housing 60 and the motor housing 70 may be separate components. The impeller housing 60 may carry the impeller assembly 65 therein. The impeller assembly 65 may include an impeller shaft 66 and an impeller 67 that rotates relative to the impeller housing 60 to drive or pump blood from the left ventricle 16 of the heart 18 of the patient (e.g., via the cannula 40) to the aorta 22 or portion of the anatomy in communication with the aorta. In some examples, the impeller shaft 66 and the impeller 67 may be integrally formed, whereas, in other examples the impeller shaft 66 and the impeller 67 may be separate components.

Rotation of the impeller 67 may cause blood to flow from a blood inlet 68 of the impeller housing 60 where the blood is received via openings at the blood inlet 80 along the cannula 40. Blood may enter the blood inlet 68 of the impeller housing 60 directly from the left ventricle 16 and/or via the cannula 40, through the impeller housing 60, and out of a blood outlet 90 formed on the impeller housing 60. As shown in FIG. 4, the blood inlet 68 may be formed on an end portion of the impeller housing 60 and the blood outlet 90 may be formed on a side portion of the impeller housing 60. In other examples, the blood inlet 68 and/or the blood outlet 90 may be formed on or at other portions of the impeller housing 60 or other components of the system 10.

The impeller housing 60 may be coupled to the cannula 40 and the cannula 40 may extend distally from the impeller housing 60, and the cannula 40 may receive blood from the left ventricle 16 and deliver the blood to the blood inlet 68. Alternatively or additionally, the impeller housing 60 may be further elongated than depicted in FIG. 4 and define all of or at least part of the cannula 40 extending into the left ventricle of the patient. Other suitable configurations of the impeller housing 60 and/or the cannula 40 are contemplated.

The motor housing 70 may carry a motor 72, and the motor 72 may be configured to rotatably drive the impeller 67 relative to the impeller housing 60 when the motor 72 is actuated. In some examples, the motor 72 may rotate a drive shaft 74 (e.g., a motor shaft) coupled with a drive or driving magnet 76 (e.g., a first magnet) within the motor housing 70. Rotation of the driving magnet 76 may cause rotation of a driven magnet 78 (e.g., a second magnet) within the impeller housing 60 that may be coupled with and/or may be part of the impeller assembly 65, wherein the driving magnet 76 and the driven magnet 78 may form or at least partially form a magnetic assembly. More specifically, in configurations incorporating the impeller shaft 66, the impeller shaft 66 (e.g., a driven shaft) and the impeller 67 may be coupled with and configured to rotate with the driven magnet 78. In other configurations, the motor 72 and/or the drive shaft 74 may be coupled to the impeller assembly 65 directly and/or via other suitable components.

The blood pump 100 of the circulatory support system 10 may have a longitudinally rigid portion to accommodate the components of the blood pump 100 (e.g., the motor, the impeller, magnets, etc.). For example, the length L1 extending along the impeller housing 60 and the motor housing 70, which may both be rigid housings, as depicted in FIG. 4, may entirely or at least partially define a rigid length or portion of the blood pump 100. A configuration of the blood pump 100 with a longer, rather than shorter, rigid length, however, can be difficult to deliver to a target location proximate a heart of a patient (e.g., at and/or across an aortic valve of the patient, etc.) due to a need to traverse tortuous anatomy (e.g., across an aortic arch and/or other suitable tortuous vessels) between a vessel entry location and the target location. To facilitate delivering the blood pump 100 through tortuous vessels to the target location and/or for other reasons, a configuration of the blood pump 100 with two or more rigid sub-lengths or sub-portions that are coupled to one another in a manner that allows for articulation of the sub-lengths or portions relative to one another may be utilized. In some examples, utilizing a configuration of the blood pump 100 that has two or more rigid sub-lengths or portions that are configured to articulate relative to one another have lengths shorter than the length L1 may reduce trauma to the patient as lower forces may be required to navigate tortuous anatomy during delivery of the blood pump 100 to the target location and may increase a patient population in which the blood pumps 100 may be used as a tortuous anatomy of a patient becomes less of a limiting factor for whether the blood pump 100 may be used in the patient.

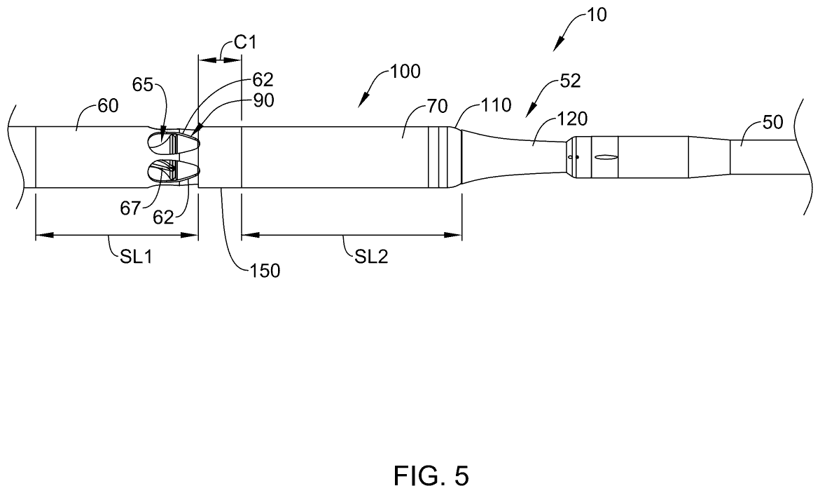

FIG. 5 is a schematic side view of a portion of the circulatory support system 10 depicting a portion of an illustrative configuration of the blood pump 100. In some examples, the blood pump 100 may include one or more connectors 150 between rigid portions of the blood pump 100 to allow the rigid portions to articulate with respect to one another. The one or more connectors 150 may be more elastomeric or flexible than the rigid portions of the blood pump 100. In some examples, the blood pump 100 may be connected to the elongate tube 50 at a junction 52. A proximal end of an impeller 67 of the impeller assembly 65 may also be visible through the outflow openings 62 of the blood outlet 90.

The junction 52 may include an end cap 110 having a proximal end region surrounding the distal end region of the elongate shaft 50, and a region of material 120 surrounding the proximal end region of the end cap 110 and extending proximally therefrom. The region of material 120 may extend in a proximal direction from the end cap 110 and surround a portion of the elongate tube 50 extending in the proximal direction from the end cap 110. The end cap 110 (e.g., as shown in FIG. 3, but not labeled) may or may not be included in the length L1.

As depicted in FIG. 5, the connector 150 may couple with and/or separate a proximal end of the impeller housing 60 (e.g., a first rigid portion of the blood pump 100) and a distal end of the motor housing 70 (e.g., a second rigid portion of the blood pump 100), such that the impeller housing 60 and the motor housing 70 may articulate with respect to one another. In some examples, the impeller housing 60 may have a first rigid sub-length or sub-portion SL1 and the motor housing 70 may have a second rigid sub-length or sub-portion SL2, where each of the first rigid sub-length SL1 and the second rigid sub-length SL2 is shorter than the rigid length L1 extending between a distal end of the impeller housing 60 and a proximal end of the motor housing 70. As discussed, reducing a continuous length of rigid portions of the blood pump 100 and allowing for the rigid portions to articulate with respect to one another may facilitate delivering and/or positioning the blood pump 100 at the target location with trauma to the patient mitigated.

The rigid lengths or portions of the blood pump 100 (e.g., the length L1, the rigid sub-lengths SL1, SL2, etc.) may be any suitable lengths. In some embodiments SL1 may have a length that is less than or equal to the length of SL2. In some examples, the rigid length L1 of the blood pump 100 may be in a range of 20 millimeters (mm) to 80 mm. In some examples, the rigid sub-length SL1 of the impeller housing 60 may be in a range of 10 mm to 20 mm. In some examples, the rigid sub-length SL2 of the motor housing 70 may be in a range of 10 mm to 60 mm. Other suitable lengths of the rigid length and/or rigid sub-lengths of the blood pump 100 are contemplated.

The connector 150 may have any suitable configuration for coupling two or more rigid portions of the blood pump 100, which allow the rigid portions coupled with the connector 150 to articulate with respect to one another. For example, the connector 150 may be and/or may include a flexible tube, a resilient tube, a spring tube, a slotted tube, an adjustable rigid tube coupled with an elongate wire, a ribbon, etc., and/or a wire having a rigid distal portion and a flexible proximal portion. In some configurations, the connector 150 may be a flexible or adjustable portion of the impeller housing 60, the motor housing 70, and/or a housing that houses both of the impeller assembly 65 and the motor assembly 75, and/or other suitable configuration.

The connector 150 may be coupled with the impeller housing 60 and the motor housing 70 in any suitable manner. For example, the connector 150 may be coupled with the impeller housing 60 and/or the motor housing 70 via one or more of an adhesive, a mechanical lock, over-molding, and/or any other suitable connecting or coupling mechanism.

The connector 150 may have any suitable length C1. In some examples, the length C1 of the connector 150 may be configured to span a gap between a proximal end of the impeller housing 60 and a distal end of the motor housing 70. In some examples, the length C1 of the connector 150 may be configured to span a length from a distal end of the impeller housing 60 to a proximal end of the motor housing 70. In some examples, the length C1 of the connector 150 (e.g., when the flexible connector 150 is a wire with a rigid distal portion and flexible proximal portion and/or in other suitable instances) may be configured to span a length of the circulatory support system 10 extending from a distal end of the impeller housing 60, along the motor housing 70 and the elongate tube 50, to a location outside of the patient. In some examples, the length C1 of the connector 150 may be configured to maintain an operative distance (e.g., a magnetically operative distance) between the driving magnet 76 (e.g., a first magnet) and the driven magnet 78 (e.g., a second magnet) while the connector 150 adjusts between a first configuration and a second configuration as the impeller assembly 65 and the motor assembly 75 articulate with respect to one another. For example, the connector 150 may have a length C1 in a range of 0 mm to 5 mm. In one example, the connector 150 may have a length C1 of 0 mm when the connector 150 is in a resting state and the impeller housing 60 and the motor housing 70 are axially aligned and at least a portion with a non-0 mm length C1 when the axes of the impeller housing 60 and the motor housing 70 are transverse to one another. Other suitable lengths C1 of the connector 150 are contemplated.

The connector 150 may be configured to facilitate articulation of the rigid portions of the blood pump 100 with respect to one another up to a maximum angle therebetween (e.g., the connector 150 may limit an angle between a central axis of the impeller housing 60 and a central axis of the motor housing 70). For example, a length, a material configuration, a thickness of material, and/or other suitable parameters may be configured to limit the angle between the central axis of the impeller housing 60 and the central axis of the motor housing 70. Example suitable maximum angles include, but are not limited to, angles in a range of up to 120 degrees with a center of the range at the central axis of the motor housing 70 (e.g., a 60-degree bend to either side of the central axis of the motor housing 70). Other suitable maximum angles are contemplated.

The connector 150 may be formed from any suitable materials. Example suitable materials include, but are not limited to, polymers, metals, rigid materials, flexible materials, resilient materials, elastomer material, shape memory materials, nickel-titanium alloys (e.g., NITINOL, etc.), silicone, silicone rubber, polypropylene, polyurethane, polydimethylsiloxane (PDMS), thermoplastic polyurethanes (TPU), and/or other suitable materials. In one example, the connector 150 may be formed from an elastomer material (e.g., an elastomer tube, etc.) and/or other suitable material configured to bias the impeller housing 60 and the motor housing 70 to an axially aligned configuration. In one example, the connector 150 may be formed from a nickel-titanium alloy configured to bias the impeller housing 60 and the motor housing 70 to the axially aligned configuration, where the connector 150 may be part of one or both of the impeller housing 60 and the motor housing 70 or a separate component from the impeller housing 60 and/or the motor housing 70.

The connector 150 may have any suitable durometer. In one example, the connector 150 may be configured to have a durometer of 80 Shore A, but other suitable durometers above or below 80 Shore A are contemplated.

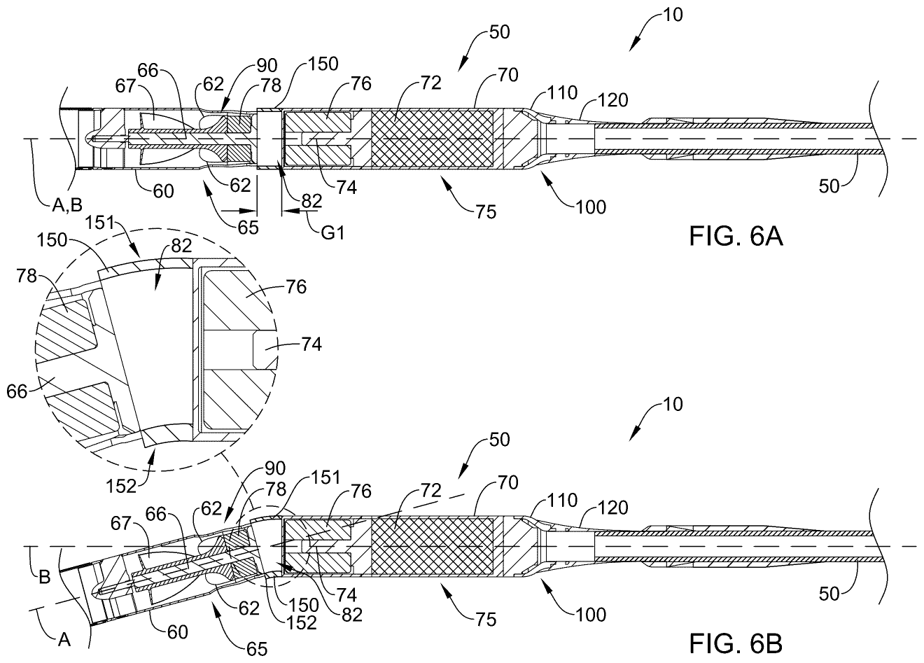

FIGS. 6A and 6B depict schematic cross-section views of the portion of the circulatory support system 10 of FIG. 5, including the junction 52 between the blood pump 100 and the elongate tube 50 of the circulatory support system 10. As depicted in FIGS. 6A and 6B, a magnetic drive assembly may couple the motor assembly 75 with the impeller assembly 65, along with the connector 150 coupling the motor housing 70 with the impeller housing 60. A gap 82 having a length G1 may span a distance between a distal end of the motor housing 70 and a proximal end of the impeller housing 60. In some examples, the forces on the impeller assembly 65 and the motor housing 70 from the connector 150 and the forces between the driving magnet 76 and the driven magnet 78 of the magnetic assembly may be configured to create and/or maintain the gap 82 to facilitate articulation of the impeller housing 60 and the motor housing 70 relative to one another.

The gap 82 may have any suitable length G1. In some examples, the gap 82 may have a length G1 configured to allow the impeller housing 60 and the motor housing 70 to articulate with respect to one another, while maintaining a magnetically operable connection between the driving magnet 76 and the driven magnet 78. Example suitable lengths G1 of the gap 82 include, but are not limited to, lengths within a range of 0 mm to 5 mm. Although the length G2 of the gap 82 is depicted in FIGS. 6A, 7A, 8A, and 8C as being greater than 0 mm, it is contemplated that the length G1 of the gap 82 may be 0 mm when the central axes of the impeller housing 60 and the motor housing 70 are aligned and the gap 82 may have at least a portion with a length that is non-0 mm when the central axes of the impeller housing 60 and the motor housing 70 are transverse to one another.

FIG. 6A schematically depicts the blood pump 100 and the connector 150 thereof in a first configuration (e.g., in a relaxed, low-energy, or pumping state or configuration), with a central axis A of the impeller housing 60 of the impeller assembly 65 aligned with a central axis B of the motor housing 70 of the motor assembly 75. Although the gap 82 is depicted in FIG. 6A, the gap 82 may be omitted when the blood pump 100 is in the first configuration such that a distal end of the motor housing 70 may be in contact with a proximal end of the impeller housing 60.

A magnetic attraction between the driving magnet 76 and the driven magnet 78 of the magnetic assembly may be configured to bias the blood pump 100 to the first configuration. Additionally or alternatively, in some examples, the connector 150 may have a spring constant or biasing force configured to bias the blood pump 100 to the first configuration. As such, the blood pump 100 may be in the first configuration when the driving magnet 76 and the driven magnet 78 are axially aligned and the connector 150 is in a neutral state.

FIG. 6B schematically depicts the blood pump 100 and the connector 150 in a second configuration (e.g., in a stressed, high-energy, or delivery state or configuration), with the central axis A of the impeller housing 60 of the impeller assembly 65 offset with respect to and/or transverse to the central axis B of the motor housing 70 of the motor assembly 75. The connector 150 may facilitate adjustment of the impeller housing 60 and the motor housing 70 to the second configuration by providing a flexible connection therebetween. The blood pump 100 may be in the second configuration with the impeller assembly 65 articulated relative to the motor assembly 75 when force (e.g., delivery forces) acting on the blood pump 100 creates a large enough moment on the blood pump 100 to overcome the magnetic force between the driving magnet 76 and the driven magnet 78 along with the biasing force, if any, of the connector 150. For example, when a distal force is acting the blood pump 100 to deliver the blood pump 100 through vasculature of the patient, the blood pump 100 may articulate to the second configuration in response to engaging curves in the vasculature. Once the distal force is removed from the blood pump 100 and/or the blood pump 100 has been positioned at a target location, the magnetic forces of the magnetic assembly and/or the bias forces of the connector 150 may automatically adjust the blood pump to the first configuration, but other suitable configurations are contemplated.

When the blood pump 100 is in the second configuration and the connector 150 spans the gap 82, the connector 150 may stretch and compress as depicted in the magnified portion of FIG. 6B. For example, when the blood pump 100 is in the second configuration, the connector 150 may include a first portion 151 that is configured to stretch and, in some cases, have a thinner cross-section than when the blood pump 100 is in the first configuration and a second portion 152 that is configured to compress and, in some cases, have a thicker cross-section than when the blood pump 100 is in the first configuration, but other suitable configurations are contemplated. In some examples, the gap 82 may have a larger longitudinal length proximate the first portion 151 of the connector 150 than a longitudinal length proximate the second portion 152 of the connector 150 when the blood pump 100 is in the second configuration. Other suitable configurations are contemplated. In some examples, an ability of the connector 150 to stretch and/or compress impacts the angle between central axes of the impeller assembly 65 and the motor assembly 75.

The driving magnet 76 and the driven magnet 78 may be configured to have any suitable magnetic force therebetween. In some examples, the driving magnet 76 and the driven magnet 78 may be configured to create a magnetic force therebetween sufficient to create and maintain a 1:1 fidelity (e.g., for each full rotation of the drive shaft 74, the impeller shaft 66 has a full rotation). In some examples, the driving magnet 76 and the driven magnet 78 may be configured to create a magnetic force therebetween that can be overcome through force acting on the blood pump 100 as the blood pump engages tortuous anatomy to allow the impeller assembly 65 to articulate relative to the motor assembly 75 without requiring a force that may damage or cause trauma to the anatomy of the patient. Example suitable operational magnetic forces between the driving magnet and the driven magnet 78 may include forces within a range of 40 gram-force to 150 gram-force. Other suitable magnetic forces which may or may not depend on a distance between the driving magnet 76 and the driven magnet 78 are contemplated.

The connector 150 may have any suitable spring constant or bias force. In some examples, the spring constant or bias force of the connector 150 may be configured to allow the connector 150 to bend when a force acting on the blood pump 100 is sufficient to overcome the magnetic force between the driving magnet 76 and the driven magnet 78 and to urge the impeller assembly 65 (e.g., the driven magnet 78) and the motor assembly 75 (e.g., the driving magnet 76) back into axial alignment after the magnetic force therebetween is overcome. In some examples, the biasing force may be less than the magnetic force between the driving magnet 76 and the driven magnet 78. Example suitable spring constants of the connector 150 may be configured to pull the driving magnet 76 and the driven magnet 78 into close enough proximity to one another to allow the driving magnet 76 and the driven magnet 78 to re-magnetically couple in an operable manner. Other suitable spring constants of the connector 150 are contemplated.

The impeller housing 60, the motor housing 70, the driving magnet 76 and/or the driven magnet 78 may be radiused to facilitate mating therebetween, smooth articulation therebetween, increasing coupling forces therebetween, increasing pushability thereof during delivery, and/or to facilitate one or more other suitable benefits. In some examples, a distal end of the motor housing 70 may have a first radius of curvature in a manner that mates with and/or is parallel with the proximal end of the impeller housing 60 that has a second radius of curvature. In some examples, a distal end of the driving magnet 76 may be radiused to facilitate magnetically mating with or being parallel with a radiused proximal end of the driven magnet 78. Although other suitable configurations are contemplated, the radiused surfaces may be part of and/or may form a ball-socket joint.

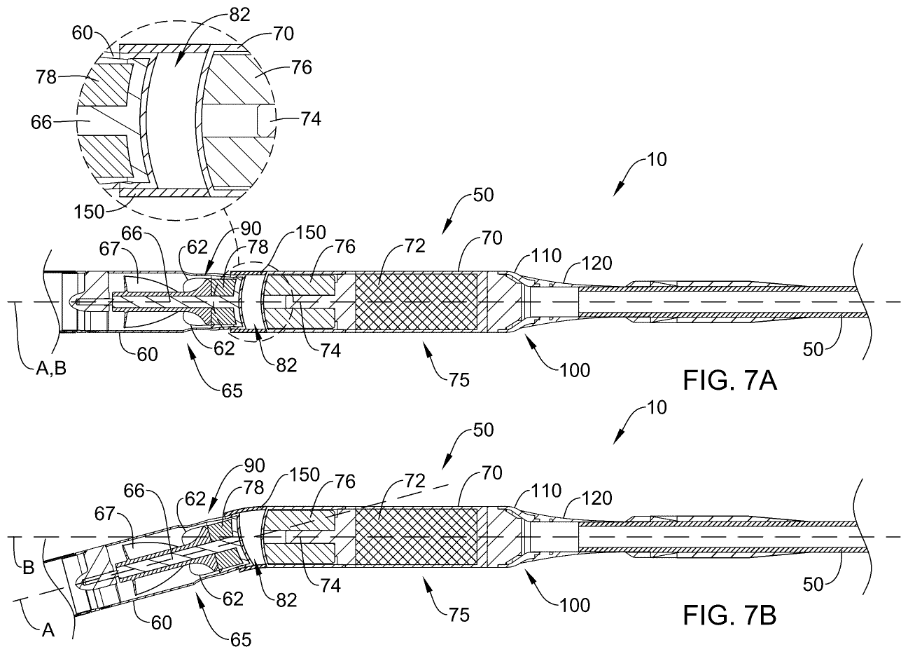

As depicted in FIGS. 7A and 7B, the distal end of the motor housing 70 may have a convex radius configured to mate with or otherwise be parallel with a concave radius of a proximal end of the impeller housing 60 and a distal end of the driving magnet 76 may have a convex radius that is configured to magnetically mate with or otherwise be parallel with a concave radius of a proximal end of the driven magnet 78. Other suitable configurations of the impeller housing 60, the motor housing 70, the driving magnet 76, and the driven magnet 78 that facilitate mating therebetween are contemplated.

FIG. 7A schematically depicts the blood pump 100 in a first configuration (e.g., in a relaxed, low-energy, or pumping state or configuration), with a central axis A of the impeller housing 60 of the impeller assembly 65 aligned with a central axis B of the motor housing 70 of the motor assembly 75 and a second radius of curvature of the proximal end of the impeller housing 60 parallel with a first radius of curvature of the distal end of the motor housing 70, as depicted in the magnified portion of FIG. 7A. Although the gap 82 is depicted in FIG. 7A with a constant longitudinal length, the gap 82 may be omitted or have a different configuration than shown when the blood pump 100 is in the first configuration such that a distal end of the motor housing 70 may be in contact with a proximal end of the impeller housing 60.

A magnetic attraction between the driving magnet 76 and the driven magnet 78 of the magnetic assembly may be configured to bias the blood pump 100 to the first configuration. In some examples, the parallel radiuses of the distal end of the driving magnet 76 and the proximal end of the driven magnet 78 may facilitate biasing the blood pump 100 to the first configuration by reducing a spacing therebetween as the blood pump 100 is adjusted to the second configuration relative to when the driving magnet 76 and the driven magnet 78 do not have radiused ends.

FIG. 7B schematically depicts the blood pump 100 in a second configuration (e.g., in a stressed, high-energy, or delivery state or configuration), with the central axis A of the impeller housing 60 of the impeller assembly 65 offset with respect to and/or transverse to the central axis B of the motor housing 70 of the motor assembly 75. With the distal end of the motor housing 70 and the proximal end of the impeller housing 60 radiused, the gap 82 may be reduced when the blood pump 100 is in the second configuration relative to when the distal end of the motor housing 70 and the proximal end of the impeller housing 60 are not radiused. The reduced length of the gap 82 when the blood pump 100 is in the second configuration may allow for utilizing a configuration of the connector 150 having a lower bias force or spring constant (e.g., a connector 150 that may be more flexible or less resilient) than when the proximal end of the impeller housing 60 and the distal end of the motor housing 70 are not radiused in a parallel manner due to the smaller gap maintaining a stronger magnetic force between the driving magnet 76 and the driven magnet 78 when the blood pump 100 is in the second configuration. Other suitable configurations are contemplated.

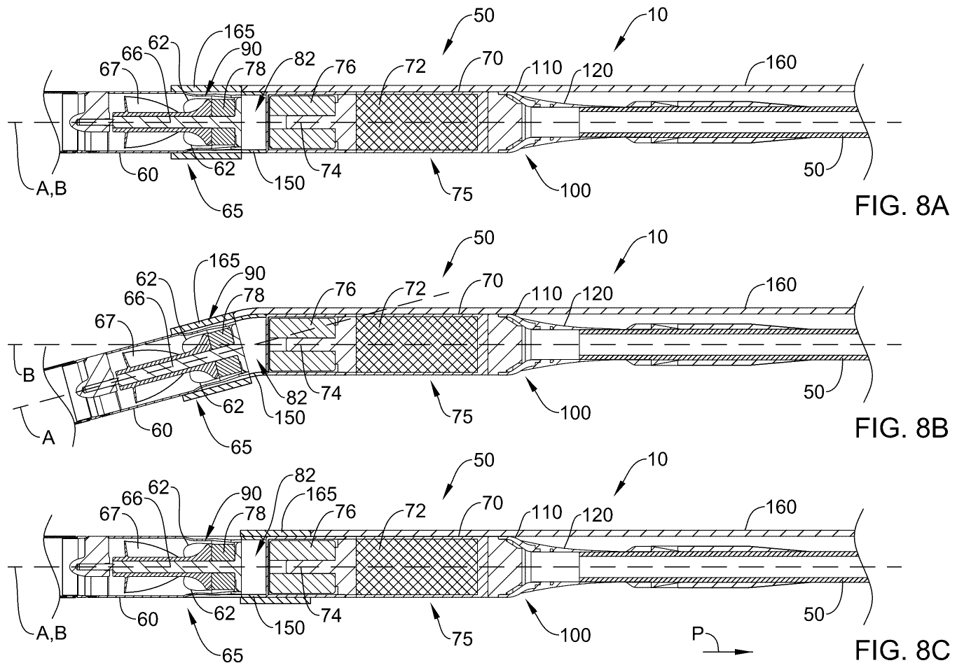

FIGS. 8A-8C depict schematic cross-section views of a portion of an illustrative circulatory support system 10, where the circulatory support system 10 may include the connector 150, a wire 160, and a tube 165 coupled with the wire 160. Although the gap 82 is depicted in FIGS. 8A and 8C, the gap 82 may be omitted when the blood pump 100 is in the first configuration and the third configuration such that a distal end of the motor housing 70 may be in contact with a proximal end of the impeller housing 60.

The wire 160 and/or the tube 165 may be adjustable or configured to be adjustable relative to the connector 150 and/or the gap 82 to facilitate allowing the impeller assembly 65 to articulate relative to the motor assembly 75 and adjust the blood pump 100 between a delivery configuration and a pumping configuration. In some configurations, the tube 165 may not include a wire and it could be constructed of a material where it functions as the wire 160. In some examples, the wire 160 may extend to a location exterior of the patient such that a user may apply a proximal force on the wire 160 to adjust the tube 165 in a proximal direction and the wire 160 may have sufficient pushability so as to be able to push the wire in a distal direction from exterior of the patient to advance the tube 165 in the distal direction. An alternative to utilizing the tube 165 may be to utilize a configuration of the wire 160 having a rigid distal portion and a flexible proximal portion, where the wire 160 may be adjustable between a position in which the rigid distal portion is distal of the connector 150 such that the impeller assembly 65 may articulate relative to the motor assembly 75 and a position in which the rigid distal portion extends along the connector 150 such that the impeller assembly 65 is prevented from articulating relative to the motor assembly 75. A wire having the distal rigid portion and the flexible proximal portion may be coupled with or relative to the impeller assembly 65 and/or the motor assembly 75 in any suitable manner. When the tube 165 and/or a wire 160 having a rigid distal portion are utilized, the connector 150 may have a spring constant or bias force biasing the blood pump 100 to the first configuration.

FIG. 8A schematically depicts the blood pump 100 in a first configuration (e.g., in a relaxed or low-energy state or configuration), with a central axis A of the impeller housing 60 of the impeller assembly 65 aligned with a central axis B of the motor housing 70 of the motor assembly 75. In the first configuration, the tube 165 may be distal of the connector 150 spanning between the distal end of the motor housing 70 and the proximal end of the impeller housing 60. When in the first configuration, the blood pump 100 may be capable of adjusting to a second configuration in which the impeller assembly 65 is articulated relative to the motor assembly 75.

FIG. 8B schematically depicts the blood pump 100 in a second configuration (e.g., in a stressed or high-energy state or configuration), with the central axis A of the impeller housing 60 of the impeller assembly 65 offset with respect to and/or transverse to the central axis B of the motor housing 70 of the motor assembly 75. In the second configuration, the tube 165 may remain distal of the connector 150 spanning between the distal end of the motor housing 70 and the proximal end of the impeller housing 60. As discussed, the blood pump 100 may enter the second configuration during delivery of the blood pump 100 to a target location and/or at one or more other suitable times.

FIG. 8C schematically depicts the blood pump 100 in a third configuration (e.g., in a relaxed or low-energy state or configuration), with the central axis A of the impeller housing 60 of the impeller assembly 65 aligned with the central axis B of the motor housing 70 of the motor assembly 75. In the third configuration, a force in a proximal direction P has been applied to the wire 160 from a location exterior of the patient and the tube 165 may be adjusted in a proximal direction so as to span the gap 82 and extend over the connector 150. As the tube 165 extends over the gap 82 and/or the connector 150, the central axis A of the impeller housing 60 may be urged into axial alignment with the central axis B of the motor housing 70. Then, when the tube 165 has been positioned over the gap 82 and/or the connector 150, the tube 165 may prevent articulation of the impeller housing 60 relative to the motor housing 70.

When in the third configuration, the blood pump 100 may be in a configuration for pumping blood through vasculature of the patient. When the blood pump 100 is to be removed from the patient, a user may apply a force on the wire 160 in a distal direction to advance the tube 165 in a distal direction (e.g., in a direction opposite of the proximal direction P) such that the blood pump 100 is adjusted to the first configuration, depicted in FIG. 8A. Once in the first configuration, the blood pump 100 may be withdrawn through the vasculature from the patient in a manner that allows the blood pump 100 to adjust between the first and second configurations.

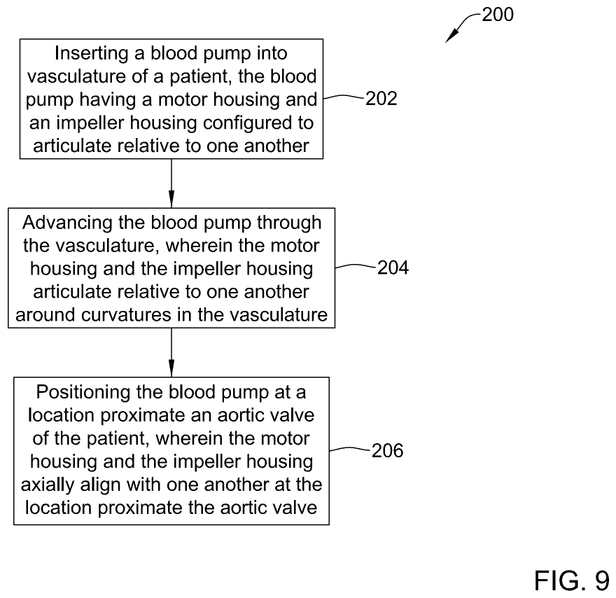

FIG. 9 depicts a schematic diagram of an illustrative method 200 of using a blood pump of a circulatory support system. The method 200 may utilize the illustrative blood pump configurations discussed herein and/or other suitable configurations of blood pumps. In some examples, the blood pump may include a motor housing and an impeller housing configured to articulate relative to one another. Although other suitable configurations are contemplated, the motor housing and the impeller housing may be coupled to one another via a magnetic coupling and/or a connector extending between and/or spanning the motor housing and the impeller housing. The magnetic coupling may be formed from a magnetic drive system configured to drive an impeller of the blood pump, where a driving magnet may be located in the motor housing and coupled to a motor in the motor housing and a driven magnet may be located in the impeller housing and coupled with the impeller in the impeller housing. The magnetic coupling may be configured to bias the motor housing and impeller housing to an axially aligned configuration. The connector may be configured to limit articulation between the motor housing and the impeller housing (e.g., limit an angle therebetween) and/or to bias the motor housing and the impeller housing to the axially aligned configuration.

The method 200 may include inserting 202 the blood pump into vasculature of a patient. In some examples, the blood pump may be inserted into the vasculature of the patient through an access site that is proximate a femoral artery of the patient. Other suitable access sites are contemplated.

The method further includes advancing 204 the blood pump through the vasculature. In some examples, a force may be applied to an elongate tube extending in a proximal direction from the blood pump to advance the blood pump through the vasculature to a target location. As the blood pump advances through the vasculature, the motor housing and the impeller housing may articulate relative to one another as a distal force is applied to the blood pump and the blood pump advances around curves in the vasculature (e.g., curvatures including, but not limited to, the aortic arch).

The method 200 may further include positioning 206 the blood pump at a target location. In some examples, the target location may be a location proximate an aortic valve of the patient. Other suitable target locations are contemplated. In one example, when positioning the blood pump at the target location, the blood pump may be positioned across the aortic valve of the patient such that blood inflow openings are located distal of the aortic valve (e.g., in the left ventricle and/or other suitable location) and blood outflow openings are located proximal of the aortic valve (e.g., in the aorta and/or other suitable location).

Once the blood pump is positioned at the target location and/or free from curvatures of the vasculature of the patient, the blood pump may return to a configuration in which the motor housing and the impeller housing are axially aligned or are in one or more other predetermined alignments. In some examples, the blood pump may automatically return to the configuration in which the motor housing and the impeller housing are axially aligned or are in one or more other predetermined alignments in response to a bias of the magnetic coupling and/or the connector. In some examples, the blood pump may return to the configuration in which the motor housing and the impeller housing are axially aligned or are in one or more other predetermined alignments in response to a user actuation (e.g., adjustment of a rigid tube or rigid portion of a wire relative to the motor housing and the impeller housing).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The scope of the disclosure is, of course, defined in the language in which the appended claims are expressed.

What is claimed is:

1. A blood pump comprising:
an impeller assembly comprising an impeller housing having a first central axis;
a motor assembly comprising a motor housing having a second central axis;
a magnetic assembly magnetically coupling the impeller assembly with the motor assembly; and
a flexible connector coupling the impeller housing with the motor housing, and
wherein the magnetic assembly is configured to bias the impeller housing and the motor housing to a first configuration with the first central axis aligned with the second central axis.

2. The blood pump of claim 1, wherein the impeller housing is rigid and the motor housing is rigid.

3. The blood pump of claim 1, wherein the flexible connector is formed from an elastomer material configured to bias the impeller housing and the motor housing to the first configuration.

4. The blood pump of claim 1, wherein the flexible connector is an elastomer tube.

5. The blood pump of claim 1, wherein the flexible connector is formed from a nickel-titanium alloy configured to bias the impeller housing and the motor housing to the first configuration.

6. The blood pump of claim 1, wherein the flexible connector is configured to facilitate the impeller housing and the motor housing adjusting to a second configuration in which the first central axis is transverse to the second central axis.

7. The blood pump of claim 6, wherein the flexible connector is configured to limit an angle between the first central axis and the second central axis when the impeller housing and the motor housing are in the second configuration.

8. The blood pump of claim 1, wherein a distal end of the motor housing has a first radius of curvature and a proximal end of the impeller housing has a second radius of curvature configured to mate with the first radius of curvature.

9. The blood pump of claim 1, further comprising:
a wire; and
a tube coupled with the wire, and
wherein the tube is configured to adjust from a first position in which the tube is distal of the flexible connector and a second position in which the tube extends between the impeller housing and the motor housing to maintain the first central axis in alignment with the second central axis.

10. The blood pump of claim 1, wherein the impeller assembly comprises an impeller within the impeller housing and the motor assembly comprises a motor within the motor housing.

11. The blood pump of claim 10, wherein the magnetic assembly comprises a first magnet within the impeller housing and coupled with an impeller shaft coupled with the impeller and a second magnet within the motor housing and coupled with a drive shaft coupled with the motor.

12. The blood pump of claim 1, wherein a flexible elongate shaft extends in a proximal direction from the motor assembly.

13. A blood pump comprising:
an impeller assembly comprising a first magnet, an impeller shaft coupled with the first magnet, an impeller coupled with the impeller shaft, and an impeller housing that houses the first magnet, the impeller shaft, and the impeller;
a motor assembly comprising a second magnet, a drive shaft coupled with the second magnet, a motor coupled with the drive shaft, and a motor housing that houses the second magnet, the drive shaft, and the motor; and
a resilient connector coupling the impeller housing with the motor housing and configured to bias the impeller housing and the motor housing to an axial configuration and allow the impeller housing and the motor housing to articulate relative to one another.

14. The blood pump of claim 13, wherein the first magnet and the second magnet are configured to bias the impeller housing and the motor housing to the axial configuration.

15. The blood pump of claim 13, wherein the resilient connector has a length configured to maintain an operative distance between the first magnet and the second magnet while the resilient connector adjusts between a first configuration and a second configuration.

16. The blood pump of claim 13, wherein the resilient connector is formed from an elastomer material.

17. The blood pump of claim 13, wherein a distal end of the motor housing has a first radius of curvature and a proximal end of the impeller housing has a second radius of curvature configured to mate with the first radius of curvature.

18. A method of using a blood pump, the method comprising:
inserting the blood pump into vasculature of a patient, the blood pump comprising a motor housing and an impeller housing configured to articulate relative to one another;
advancing the blood pump through the vasculature, wherein the motor housing and the impeller housing are configured to articulate relative to one another around curvatures in the vasculature; and
positioning the blood pump at a location proximate an aortic valve of the patient, wherein the motor housing and the impeller housing are configured to axially align with one another at the location proximate the aortic valve.

19. The method of claim 18, wherein the motor housing and the impeller housing are coupled to one another via a magnetic coupling and the magnetic coupling biases the motor housing and the impeller housing to an axially aligned configuration.

20. The method of claim 18, wherein the blood pump comprises a flexible connector coupled with the motor housing and the impeller housing, the flexible connector is configured to limit articulation between the motor housing and the impeller housing.
